SEQUENCE LISTING (4 sequences)

SEQ ID NO: 1 - DNA, 18 bp, Artificial Sequence
  oligonucleotide probe
accatagcgctcgctggt

SEQ ID NO: 2 - DNA, 18 bp, Artificial Sequence
  oligonucleotide probe
gtggcccacgacagcttt

SEQ ID NO: 3 - DNA, 18 bp, Artificial Sequence
  oligonucleotide probe
ggtcgatgcccagctccc

SEQ ID NO: 4 - DNA, 18 bp, Artificial Sequence
  oligonucleotide probe
gtcaaagccttgtttgtc

(19) United States
(12) Patent Application Publication
(10) Pub. No.: US 2004/0009519 A1
(43) Pub. Date: Jan. 15, 2004

(54) Method for detecting protozoa of the genus Naegleria

(76) Inventors: Jiri Snaidr, Grobinzemoos (DE); Karlheinz Trebesius, Bad Endorf (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/442,034

(22) Filed: May 20, 2003

Related U.S. Application Data
(63) Continuation of application No. PCT/EP01/13625, filed on Nov. 22, 2001.

(30) Foreign Application Priority Data
Nov. 22, 2000 (DE) 100 57 841.1

(51) Int. Cl. C12Q 1/68; C07H 21/04

(52) U.S. Cl. 435/6; 536/23.7

(57) ABSTRACT

The invention relates to a method for rapidly and specifically detecting protozoa of the genus Naegleria and especially the genus Naegleria fowleri. The invention further relates to specific oligonucleotide probes that are used in the detection method and kits containing said oligonucleotide probes.

### Related Applications

This Application is a continuation of the International Application PCT/EP01/13625 filed Nov. 22, 2001 and published in German as WO 02/42492, which claims the benefit of priority of German Application DE 100 57 841.1 filed Nov. 22, 2000, both of which are expressly incorporated herein by reference in their entireties.

### Background of the Invention

1. Field of the Invention

The invention relates to a method for rapidly and specifically detecting protozoa of the genus Naegleria and especially of the species Naegleria fowleri. The invention further relates to specific oligonucleotide probes that are used in the detection method and to kits containing said oligonucleotide probes.

2. Description of the Related Art

Naegleriae are small, free-living, flagellated amoebae with worldwide distribution which are found predominantly in water samples. Of the Naegleria species known today, only Naegleria fowleri is known to be pathogenic. Naegleria fowleri exists in three forms: cyst, amoeba and flagellate. In warm water, the protozoan may transform rapidly from the amoebic to the flagellated stage, which enables a fairly rapid mode of locomotion. Infection with these free-living amoebae presumably takes place during swimming and diving in fresh water (particularly in calm, warm ponds or lakes, sometimes also in poorly maintained whirlpools). The amoebae invade nasally, migrate to the brain through the olfactory nerve and produce a toxin, which destructs the brain. The organism does not form cysts in the human organism. This specific disease is called primary amoebic meningoencephalitis (PAM). It progresses rapidly and is almost always lethal. PAM occurs mainly in children or younger adults who appear completely healthy before occurrence of PAM. Sudden disease onset is characterized by fever, nausea, vomiting, headaches and stiff neck. The disease progresses rapidly with the clinical picture of a pyrogenic meningoencephalitis; the patients become comatose and frequently die within 1 week, without apparent neurological focal symptoms. In many cases, diagnosis is made post mortem after brain autopsy. The single, currently known treatment option is the systemic and intrathecal administration of high doses of amphotericin B and miconazole in combination with the oral administration of rifampicin, and the administration of amphotericin B together with metronidazole.

The very rapid diagnosis of the infectious microbes is the prerequisite to start treatment in time. Naegleria australiensis may also possess pathogenic properties, which are, however, weaker than those of Naegleria fowleri.

Analysis of human samples, bathing areas, swimming pools, indoor swimming pools and whirlpools for the presence of Naegleria species therefore would be extremely important, and of great public interest in accordance with a preventive health strategy. Therefore, there is a need for fast and precise identification methods to detect Naegleria species and to distinguish pathogenic from non-pathogenic species.

Today, several approaches exist to identify Naegleria fowleri. These include serological tests with specific monoclonal antibodies (Visvesvara et al. 1987 J Clin Microbiol 25:1629-1634), electrophoretic profiles of isoenzymes (De Jonckheere 1981 Protistologica XVII:423-429), characterization of DNA restriction fragment length polymorphisms (RFLPs) (McLaughlin et al. 1988 J Clin Microbiol 26:1655-1658) or by polymerase chain reaction (PCR) (Sparagano 1993 FEMS Microbio. Lett 110:325-330). However, these methods are very elaborate, time consuming and depend on a high concentration of amoebae. Furthermore, they frequently demonstrate significant lack of specificity, and are therefore not suited for robust, highly specific and rapid analysis. Another possibility may be DNA probes for species-specific detection of Naegleriae after cultivation and using dot-blot hybridization (Kilvington et al. 1995 Appl Env Microb 61:2071-2078). Apart from the fact that a time consuming cultivation step is necessary in this method, followed by nucleic acid hybridization, the probes known in the prior art are usually, due to their length of several hundred bases, not suitable for the use in in situ or whole cell hybridizations.

### SUMMARY OF THE INVENTION

It is an object of the present invention to provide probe sequences and a method for rapid and specific detection and, if required or desired, for visualization of protozoa of the genus Naegleria.

According to the invention, this problem is solved by providing the following nucleic acid probe molecules:

a) oligonucleotide molecules detecting all species of the genus Naegleria (i.e. N. australiensis, N. italica, N. jamiesoni, N. andersoni, N. lovanensis, N. fowleri, N. gruberi, N. clarki and N. minor):

| | | |
|---|---|---|
| NAEG1: | 5′-ACC-ATA-GCG-CTC-GCT-GGT-3′ | (SEQ ID NO:1) |
| NAEG2: | 5′-GTG-GCC-CAC-GAC-AGC-TTT-3′ | (SEQ ID NO:2) |

b) oligonucleotide molecules specifically detecting the species Naegleria fowleri:

| | | |
|---|---|---|
| NFOW1: | 5′-GGT-CGA-TGC-CCA-GCT-CCC-3′ | (SEQ ID NO:3) |
| NFOW2: | 5′GTC-AAA-GCC-TTG-TTT-GTC-3′. | (SEQ ID NO:4) |

Further subject of the invention are modifications of the oligonucleotides NAEG1, NAEG2, NFOW1 and NFOW2 demonstrating a specific hybridization with nucleic acid sequences of Naegleria species despite variations in sequence and/or length.

These especially include:

a) nucleic acid molecules (i) being identical to the oligonucleotide sequences NAEG1, NAEG2, NFOW1 or NFOW2 to at least 60%, 65%, preferably to at least 70%, 75%, more preferably to at least 80%, 84%, 87% and particularly preferred to at least 90%, 94%, 97% of the bases (wherein the sequence region of the nucleic acid molecule corresponding to the sequence region of NAEG1, NAEG2, NFOW1 and NFOW2 is to be considered and not the entire sequence of a nucleic acid molecule which possibly may be extended by one or multiple bases compared to NAEG1, NAEG2, NFOW1 and NFOW2) or (ii) distinguishing from NAEG1, NAEG2, NFOW1 or NFOW2 by at least one deletion and/or addition, and which render possible a specific hybridization with nucleic acid sequences of Naegleria species. “Specific hybridization” hereby means that under the here described hybridization conditions or those known to the person skilled in the art in relation to in situ hybridization techniques, only the ribosomal RNA of the target organisms (i.e. for example of N. fowleri regarding the oligonucleotide NFOW1) binds to the oligonucleotide but not to the rRNA of non-target organisms (e.g., of N. lovaniensis regarding the probe NFOW1).

b) Nucleic acid molecules being complementary to the nucleic acid molecules mentioned in a) or to the probes NAEG1, NAEG2, NFOW1 or NFOW2, or which specifically hybridize with the nucleic acid molecules mentioned in a) or with the probes NAEG1, NAEG2, NFOW1 or NFOW2;

c) Nucleic acid molecules comprising the oligonucleotide sequences NAEG1, NAEG2, NFOW1, NFOW2 or the sequence of a nucleic acid molecule according to a) or b), having at least one further nucleotide in addition to the mentioned sequences or their modifications according to a) or b), and allowing a specific hybridization with nucleic acid sequences of Naegleria species.

### DETAILED DESCRIPTION OF THE PREFERRED EMBODIMENT

The degree of sequence identity of a nucleic acid molecule to the probes NAEG1, NAEG2, NFOW1 and NFOW2 can be determined using the usual algorithms. In this respect, e.g., the program for determining the sequence identity available under www.ncbi.nlm.nih.gov/BLAST (on this page e.g. the link “Standard nucleotide-nucleotide BLAST [blastn]”) is suitable.

The nucleic acid molecules according to the invention which may be used as probes within the scope of the detection of Naegleria species, comprise synthetically produced probe molecules as well as recombinantly generated probes with the above denoted probe sequences. Also, the actual nucleotides may be replaced at the non-discriminatory positions by nucleotide analogues such as inosine and the like. Furthermore, the probe molecules may also be synthesized using nucleotide analogues such as PNA (peptide nucleic acids) and the like.

Accordingly, the oligonucleotide molecules mentioned and the modifications of these molecules according to the invention are used in an inventive method for detecting microorganisms in a sample using a nucleic acid probe, the method comprising the following steps:

a) fixing the Naegleria cells present in the sample

b) incubating the fixed cells with at least one of the nucleic acid probe molecules according to the invention, particularly with a nucleic acid probe molecule selected from the group consisting of the molecules:

| | |
|---|---|
| 5′-ACC-ATA-GCG-CTC-GCT-GGT-3′, | (SEQ ID NO:1) |
| 5′-GTG-GCC-CAC-GAC-AGC-TTT-3′, | (SEQ ID NO:2) |
| 5′-GGT-CGA-TGC-CCA-GCT-CCC-3′, and | (SEQ ID NO:3) |
| 5′-GTC-AAA-GCC-TTG-TTT-GTC-3′, | (SEQ ID NO:4) |

in order to achieve hybridization,

c) removing non-hybridized nucleic acid probe molecules;

d) detecting and, optionally, quantifying and visualizing the Naegleria cells with hybridized nucleic acid probe molecules.

Within the scope of the present invention, “fixing” of the cells is meant to be a treatment with which the cell envelope of the protozoa is made permeable for nucleic acid probes. The nucleic acid probes consisting of an oligonucleotide and a marker linked thereto are then able to penetrate the cell envelope in order to bind to the target sequence corresponding to the nucleic acid probe in the cell. The bonding is to be conceived as formation of hydrogen bonds among complementary nucleic acid regions. The envelope may be a lipid envelope coating a virus, the cell wall of bacteria or the cell wall of a protozoan. For fixation, a low percentage paraformaldehyde solution or a diluted formaldehyde solution may generally be used. If the cell wall can not be penetrated by the nucleic acid probes using these techniques, the expert will know sufficient further techniques leading to the same result. These include for example ethanol, methanol, mixtures of these alcohols, enzymatic treatments or the like.

The nucleic acid probe within the spirit of the invention may be a DNA or RNA probe comprising usually between 12 and 1000 nucleotides, preferably between 12 and 500, more preferably between 12 and 200, especially preferably between 12 and 50 and between 15 and 40, and most preferably between 17 and 25 nucleotides. The selection of the nucleic acid probes is done according to the criteria of whether a complementary sequence is present in the microorganism to be detected. By selecting a defined sequence, a bacteria species, a bacteria genus or an entire bacteria group may be detected. In a probe consisting of 15 nucleotides, 100% of the sequence should be complementary. In oligonucleotides of more than 15 nucleotides, one or more mismatches are allowed. By compliance with stringent hybridization conditions, it is guaranteed that the nucleic acid probe molecule indeed hybridizes with the target sequence. Moderate conditions according to the spirit of the invention are e.g., 0% formamide in a hybridization buffer such as the one described in example 1. Stringent conditions according to the spirit of the invention are e.g. 20-80% formamide in the hybridization buffer.

The nucleic acid probe may hereby be complementary to a chromosomal or episomal DNA, but also to an mRNA or rRNA of the microorganism to be detected. Within the scope of the present invention, the nucleic acid probe is preferably complementary to the 18S RNA of the Naegleria species to be detected. It is advantageous to select a nucleic acid probe that is complementary to a region present in copies of more than 1 in the microorganism to be detected. The sequence to be detected is preferably present in 500-100,000 copies per cell, especially preferred in 1,000-50,000 copies. For this reason, the rRNA is used preferably as target site, since in each active cell the ribosomes as sites of protein biosynthesis are present in many thousand copies.

According to the invention, the nucleic acid probe is incubated with the microorganism fixed in the above sense, in order to allow penetration of the nucleic acid probe molecules into the microorganism and hybridization of nucleic acid probe molecules with the nucleic acids of the microorganisms. Then, usual washing steps remove the non-hybridized nucleic acid probe molecules. The specifically hybridized nucleic acid probe molecules can then be detected in the respective cells, on condition that the nucleic acid probe is detectable, e.g., the probe molecule being linked to a marker by covalent binding. As detectable markers, fluorescent groups such as CY2 (available from Amersham Life Sciences, Inc., Arlington Heights, USA), CY3 (also available from Amersham Life Sciences), CY5 (also available from Amersham Life Sciences), FITC (Molecular Probes Inc., Eugene, USA), FLUOS (available from Roche Diagnostics GmbH, Mannheim, Germany), TRITC (available from Molecular Probes Inc. Eugene, USA) or FLUOS-PRIME are used, which are all well known to the person skilled in the art. Chemical markers, radioactive markers or enzymatic markers such as horseradish peroxidase, acid phosphatase, alkaline phosphatase, peroxidase may be used as well. For each enzyme of this series, a number of chromogens is known which may be converted instead of the natural substrate, and may be transformed to either colored or fluorescent products. Examples of such chromogens are listed in the subsequent Table:

TABLE

| Enzymes | Chromogen |
|---|---|
| 1. Alkaline phosphatase | 4-methylumbelliferyl phosphate (*), |
| and acid phosphatase | bis(4-methylumbelliferyl phosphate), |
| | (*) 3-O-methylfluorescein, flavone-3- |
| | diphosphate triammonium salt (*), |
| | p-nitrophenylphosphate disodium salt |
| 2. Peroxidase | tyramine hydrochloride (*), 3-(p- |
| | hydroxyphenyl)-propionate (*), |
| | p-hydroxyphenethyl alcohol(*), |
| | 2,2′-azino-di-3-ethylbenzothiazoline |
| | sulfonic acid (ABTS), ortho- |
| | phenylendiamine dihydrochloride, |
| | o-dianisidine, 5-aminosalicylic acid, |
| | p-ucresol (*), 3,3′-dimethyloxy benzidine |
| | 3-methyl-2-benzothiazoline hydrazone, |
| | tetramethylbenzidine |
| 3. Horseradish peroxidase | H₂O₂ + diammonium benzidine |
| | H₂O₂ + tetramethylbenzidine |
| 4. β-D-galactosidase | o-nitrophenyl-β-D-galactopyranoside, |
| | 4-methylumbelliferyl-β-D-galactoside |
| 5. Glucose oxidase | ABTS, glucose and thiazolyl blue |

*fluorescence

Finally it is possible to generate the nucleic acid probe molecules in such a matter that another nucleic acid sequence suitable for hybridization is present at their 5' or 3' ends. This nucleic acid sequence comprises again approx. 15 to 1,000, preferably 15-50 nucleotides. This second nucleic acid part may be again detected by an oligonucleotide probe detectable by one of the above mentioned agents.

Another possibility is the coupling of the detectable nucleic acid probe molecules to a hapten, which may subsequently be brought in contact with a hapten-recognizing antibody. Digoxigenin may be named as an example for such a hapten. Apart from the described examples, other examples are also well known to the expert.

The standard hybridization procedure is performed on slides, on filters, on a microtitre plate, or in a reaction vessel. The analysis depends on the kind of labelling of the used probe and may be conducted using an optical microscope, epifluorescence microscope, chemiluminometer, fluorometer, flow cytometer, etc.

The probe molecules according to the invention may be used within the scope of the detection method with various hybridization solutions. Various organic solvents may be used in concentrations of 0% to 80%. For example, formamide is used preferably in a concentration of 20% to 60%, especially preferred in a concentration of 20% in the hybridization buffer. Furthermore, a salt, preferably sodium chloride, is contained in the hybridization buffer in a concentration of 0.1 mol/L to 1.5 mol/L, preferably of 0.5 mol/L to 1.0 mol/L and more preferably of 0.7 mol/L to 0.9 mol/L and most preferably of 0.9 mol/L. For buffering of the hybridization buffer, various compounds such as Tris-HCl, sodium citrate, PIPES or HEPES buffer may be used in a range of 0.01 mol/L and 0.1 mol/L, preferably between 0.01 mol/L and 0.08 mol/L and especially preferred as 0.02 mol/L. The pH usually lies between 6.0 and 9.0, preferably between 7.0 and 8.0. Preferably, the hybridization buffer contains 0.02 mol/L Tris-HCl, pH 8.0.

In addition detergents such as Triton X or sodium dodecyl sulfate (SDS) are usually present in a concentration of 0.001% to 0.2%, preferably of 0.05% to 0.1%. Here, an especially preferred hybridization buffer contains 0.01% SDS.

Further additives may be used in various situations, such as unlabelled nucleic acid fragments (e.g., fragmented salmon sperm DNA, unlabelled oligonucleotides, and the like), or molecules, which may lead to an acceleration of the hybridization reaction due to a limitation in the reaction space (polyethylene glycol, polyvinyl pyrrolidone, dextran sulfate, and the like). The expert may add such additives in the known and usual concentrations to the hybridization buffer.

It shall be understood that the expert can choose the listed concentrations of the constituents of the hybridization buffer in such a way that the desired stringency of the hybridization reaction is achieved. Especially preferred embodiments reflect stringent to particularly stringent hybridization conditions. Using these stringent conditions, the expert can find out if a particular nucleic acid molecule enables the specific detection of nucleic acid sequences of Naegleria species, and may therefore be used reliably within the scope of the invention.

The concentration of the probe may vary greatly, depending on the marker and number of the target structure to be expected. In order to allow rapid and efficient hybridization, the probe number should exceed the number of the target structures by several orders of magnitude. However, it needs to be observed that in fluorescence in situ hybridization (FISH), high levels of fluorescence-labelled hybridization probe results in increased background fluorescence. The probe amount should therefore be between 0.5 ng/μl and 500 ng/μl, preferably between 1.0 ng/μl and 100 ng/μl and especially preferred at 50 ng/μl.

The hybridization is followed by a stringent washing step, which is intended to remove any unspecifically bound probe molecules. Hereby, buffer solutions are used which can in principle be very similar to the hybridization buffer (buffered sodium chloride solution), except that the washing step is performed in a buffer with lower salt concentration or at higher temperatures.

For theoretical estimation of the hybridization conditions, the following formula may be used:

Td=81.5+16.6 lg[Na⁺]+0.4×(% GC) 820/n−0.5×(% FA)

Td =dissociation temperature in ° C.

[Na⁺] =molarity of the sodium ions

% GC =percentage of guanine and cytosine nucleotides relative to the number of total bases

n=hybrid length

% FA=percentage of formamide.

Using this formula, the formamide content (which should be as low as possible due to its tokicity) of the washing buffer may, for example, be replaced by a correspondingly lower sodium chloride content.

In the preferred embodiment of this invention, the sodium chloride content of the washing buffer is from 0.014 mol/L to 0.9 mol/L, especially preferably 0.225 mol/L, with 0.02 mol/L Tris-HCl, pH 8.0 and 0.01% SDS, and with 0-0.005 mol/L EDTA, especially preferably 0 mol/L EDTA.

However, concerning the in situ hybridization methods, the person skilled in the art knows from the extensive literature that and in which way the named contents can be varied.

The same applies to the stringency of the hybridization conditions, as outlined above for the hybridization buffer.

In an alternative embodiment of the method according to the invention, the nucleic acid probe molecules according to the invention are used in the so-called Fast-FISH method for specifically detecting Naegleria species. The Fast-FISH method is known to the expert and is, for example, described in the German patent application DE 199 36 875.9 and in the international application WO 99/18234. Hereby it is expressly referred to the disclosure contained in these documents for performing the there described detection procedures.

An important advantage of the method described in this application for the specific detection of Naegleria species compared to conventional detection methods is its speed. Since death by infection with Naegleria fowleri occurs within a few days, fast and, above all, specific detection is imperative in order to be able to administer suitable therapeutics (e.g. amphotericin B) in time. So far, diagnosis is made primarily by a post mortem brain autopsy due to the slowness of conventional methods.

Another advantage is the specificity of this method. With the used gene probes, all species of the genus Naegleria can be specifically detected and visualized, but it is also possible to detect and visualize highly specifically only the pathogenic species Naegleria fowleri. By visualization of Naegleriae, a visual control may be performed at the same time.

Another advantage of this method is that it may optionally be performed without cultivation.

Using the method, large sample numbers can be tested easily for the presence of Naegleria cells, and particularly for presence of Naegleria fowleri.

The variety of labelling options enables also the concurrent detection of two or more overlapping or non-overlapping populations. By using for example two different fluorescence markers, Naegleria fowleri can thus be detected specifically in the background of all other cells belonging to the genus Naegleria.

The method according to the invention may be used variously. Environmental samples can be tested for the presence of Naegleriae. These samples may be collected from air, water or soil.

Another field of applying the method according to the invention is the analysis of food. This includes, above all, foods mixed with water.

The method according to the invention can also be used for the analysis of medical samples. It is suitable for the analysis of tissue samples such as biopsy material from brain, lung, tumors or inflammatory tissue, from secretions such as sweat, saliva, semen and nasal secretions, urethra or vaginal discharges as well as for urine and stool samples.

Another example for the application of the present method is the analysis of lakes and rivers, such as bathing areas.

Furthermore, according to the invention, a kit for performing the method for fast and highly specific detection of Naegleria cells in a sample is provided. The kit comprises as its main component an oligonucleotide probe that is specific for the microorganism to be detected. It further comprises a hybridization buffer and a washing buffer. The selection of the hybridization buffer depends primarily on the length of the used nucleic acid probes. Examples for hybridization conditions are described in Stahl & Amann 1991, in: Stackebrandt and Goodfellow (eds.), Nucleic Acid Techniques in Bacterial Systematics; John Wiley & Sons Ltd., Chichester, UK). The kit contains at least one of the above-mentioned specific probes for detection of Naegleriae, preferably it contains at least one probe which is suitable for detection of all species of the genus Naegleria, i.e. preferably NAEG1 or NAEG2, and at least one probe which is suitable for the specific detection of the species Naegleria fowleri, i.e. NFOW1 or NFOW2.

The following example is intended to describe the invention, however, without limiting it.

### EXAMPLE

### Detection of Naegleriae in a Water Sample

A water sample is centrifuged, and 1/10 volume of an at least 37% containing paraformaldehyde solution (Merck, Darmstadt, Germany) is added to the pellet and mixed well. The suspension is incubated for 5 minutes at room temperature. Then, the cells are centrifuged for 5 min at 1,300 g, the supernatant is discarded, and the pellet is dissolved in an appropriate volume of 1×PBS (NaₓPO₄). Here, the volumes can be chosen freely, whereas, however, volumes are preferred that fit well into an Eppendorf reaction vessel and that can be centrifuged well, such as 100 -500 μl. After complete resuspension of the pellet, the same volume of absolute ethanol is added. In this form, the Naegleriae are storable at −20° C. for at least 3 months.

For hybridization, a suitable aliquot of the fixed cells (such as 8-10 μl) is applied onto a slide. For this, the Naegleria cells may be mixed individually or mixed with other Naegleria species or Acanthamoeba species or bacteria species.

Hybridization of the Naegleriae is performed without the increasing ethanol concentration series for permeabilization of the cell membranes, which is otherwise common according to the state of the art.

Hybridization is performed with the above-mentioned probes NAEG1 or NAEG2 for detection of amoebae of the genus Naegleria (N. fowleri, N. gruberi, N. clarki, N. australiensis, N. lovanensis, N. jamiesoni, N. italica, N. andersoni, and N. minor) or with the also above mentioned probes NFOW1 or NFOW2 for detection of the amoeba N. fowleri which is a pathogen for humans. The probes are used in a concentration of 5 ng/μl; generally a concentration between 1 and 100 ng/μl is suitable.

Several microliters of the fixed Naegleria cells are applied to a slide and dried for 20 min. Then, several microliters of a hybridization buffer (0.9 mol/L NaCl, 0.02 mol/L Tris/HCl, 0.01% SDS, 20% formamide) are applied to the well, and then incubated for 90 min at 46° C. in a humid chamber. After that, the slides are removed from the chamber, rinsed shortly with washing buffer (0.225 mol/L NaCl, 0.02 mol/L Tris, 0.01% SDS), and washed stringently in this buffer at 48° C. for 15 min. Here, stringent means that the washing conditions, as described above in detail, are selected in a way. that the target nucleic acid is still included, whereas the closest-related non-target nucleic acids are not included, i.e. are washed off.

The slides are then rinsed with distilled water and air-dried.

Optionally, unspecific staining of nucleic acid with the dye DAPi (4',6-diamidino-2-phenylindole-dihydrochloride; Sigma; Deisenhofen, Germany) may be performed in addition. For this, the samples are overlaid with a PBS solution containing 1 μg/ml DAPI and are incubated for 5-15 min in the dark at room temperature. After a further washing step with distilled water, the samples can then be analyzed in an appropriate embedding medium (Citifluor AF1, Citifluor Ltd., London, UK; Vectashild, Vector laboratories, Burlingame, USA) using a-fluorescence microscope.

What is claimed is:

1. An oligonucleotide, comprising a nucleotide sequence selected from the group consisting of:
5′-ACC-ATA-GCG-CTC-GCT-GGT-3′,
5′-GTG-GCC-CAC-GAC-AGC-TTT-3′,
5′-GGT-CGA-TGC-CCA-GCT-CCC-3′ and
5′-GTC-AAA-GCC-TTG-TTT-GTC-3′.

2. A method for detecting protozoa of the genus Naegleria in a sample, comprising:
a) fixing the Naegleria cells present in the sample;
b) incubating the fixed cells with at least one oligonucleotide selected from the group consisting of:
(i) oligonucleotides according to claim 1,
(ii) oligonucleotides being identical to at least 60% to the oligonucleotides according to claim 1 and render possible specific hybridization with nucleic acid sequences of Naegleria cells,
(iii) oligonucleotides, which distinguish from the oligonucleotides according to claim 1 by a deletion and/or addition, and render possible specific hybridization with nucleic acid sequences of Naegleria cells, and
(iv) oligonucleotides hybridizing with the above oligonucleotides under stringent conditions, in order to achieve hybridization;
c) removing non-hybridized oligonucleotides; and
d) detecting the Naegleria cells with hybridized oligonucleotides.

3. The method of claim 2, wherein the oligonucleotide is covalently linked to a detectable marker selected from the group consisting of:
a) fluorescent marker,
b) chemoluminescent marker,
c) radioactive marker,
d) enzymatically active groups,
e) hapten, and
f) nucleic acids detectable by hybridization.

4. The method according to claim 2 or 3, wherein the sample is an environmental sample and is collected from water, soil or air.

5. The method according to claim 2 or 3, wherein the sample is a food sample.

6. The method according to claim 2 or 3, wherein the sample is a medical sample.

7. The method according to claim 2, wherein detection is performed by epifluorescence microscopy.

8. The method according to claim 2, wherein detection is performed by flow cytometry.

9. The method according to claim 2, wherein the Naegleria cells are cells of the species Naegleria fowleri.

10. The method of claim 2, further comprising quantifying and visualizing the Naegleria cells with hybridized oligonucleotides.

11. A kit for for detecting protozoa of the genus Naegleria in a sample, comprising at least one oligonucleotide selected from the group consisting of:
i) oligonucleotides according to claim 1,
ii) oligonucleotides being identical to at least 60% to the oligonucleotides according to claim 1 and render possible specific hybridization with nucleic acid sequences of Naegleria cells,
iii) oligonucleotides, which distinguish from the oligonucleotides according to claim 1 by a deletion and/or addition and render possible specific hybridization with nucleic acid sequences of Naegleria cells, and
iv) oligonucleotides hybridizing with the above-mentioned oligonucleotides under stringent conditions.

12. The kit according to claim 11, further comprising a hybridization solution and a washing solution.
